(19) United States
(12) Patent Application Publication
(10) Pub. No.: US 2007/0010792 A1
(43) Pub. Date: Jan. 11, 2007

(54) Disposable aid to urination

(75) Inventor: Takauki Kimura, Woodinville, WA (US)

Correspondence Address: BLACK LOWE & GRAHAM, PLLC, 701 FIFTH AVENUE, SUITE 4800, SEATTLE, WA, 98104, US

(73) Assignee: Mega Great Empire Trading Company

(21) Appl. No.: 11/165,890

(22) Filed: Jun. 24, 2005

(51) Int. Cl. A61F 5/44

(52) U.S. Cl. 604/349; 604/329

(57) ABSTRACT

A system for collecting urine includes a fitment for receiving a funnel in sealing engagement. The fitment includes a generally cylindrical tube has a circumferential surface, a first and a second end, and defining a bore having a bore interior surface. The first end is configured to receive a funnel in the bore; the funnel in frictional engagement with the bore interior surface. The second end is configured to engage lips of a bladder at a lip interior surface in sealing engagement and thereby configured to conduct a liquid through the funnel to the bladder.

### SUMMARY OF THE INVENTION

An embodiment of the invention includes a urine collection device designed to enable women to urinate while standing. Advantageously, the urinary device provides an hygienic solution to challenges often experienced by women in hospital, clinic, home health and nursing care settings. Facilitating standing urination has proven useful for patients who have had hip replacement surgery or are restricted from sitting for periods of time. Additionally, the urinary device facilitates the collection of urine from women thereby to obtain a “clean catch” for purposes of laboratory analysis. Catheterization may be eliminated in some situations where clean catches have proved impossible to obtain.

An embodiment of the inventive system includes a disposable, biodegradable paperboard “funnel” which connects quickly, securely, and easily to a disposable gusseted bladder. Collected urine drains into and through the funnel into the bladder where it is safely collected. A provided cap can be screwed onto the bladder's opening to secure the contents and to make is safe for handling and shipping, or for storing or disposal.

In an embodiment of the inventive system, the bladder is connected to the funnel by means of a plastic fitment configured to act as an interface between the funnel and the bladder.

A threaded interior throat provides for a secure connection between the paperboard funnel and the collector bladder sufficient to support the weight of the collected urine without mishap. The threading also creates a “score” on paperboard edges of the paperboard funnel, engaging and keeping the funnel securely within the fitment and bladder. The resulting angle between the fitment and a collecting port of the funnel is ergonomically advantageous to best accommodate the natural “draining” angles while standing.

An embodiment of the fitment includes external threads to accept protective cap. Further embodiments of the fitment include external threading to accept standard-sized covers. The screw-on cover seals the contents into the bladder. Once the urine is collected, the funnel readily detaches from the fitment for disposal, leaving only the bladder and its contents to be disposed of, or sealed and shipped to the laboratory.

The present invention comprises a system for collecting urine including a fitment for receiving a funnel in sealing engagement. The fitment includes a generally cylindrical tube having a circumferential surface, a first and a second end, and defining a bore having a bore interior surface. The first end is configured to receive a funnel in the bore; the funnel is in frictional engagement with the bore interior surface. The second end is configured to engage lips of a bladder at a lip interior surface in sealing engagement and thereby configured to conduct a liquid through the funnel to the bladder.

In accordance with further aspects of the invention, the fitment includes at least one circumferential rib configured to engage at least one linear ridge formed on the lip interior surface. In some embodiments, at least one circumferential rib may be frictionally engaged with the at least one linear ridge or alternatively the at least one circumferential rib may be fusedly attached to the at least one linear ridge.

In accordance with other aspects of the invention, the fitment includes at least one axial fin configured to engage at least one vertex of the lips in order to maintain registration between the fitment and the lips.

In accordance with still further aspects of the invention, an internal land is disposed on the first internal surface generally at the first end, the internal land being configured to engage a funnel. The internal land is further configured to engage the funnel to sealingly engage flexible panels of the funnel against a fitment rim. In alternate embodiments, the internal land may be either circularly or spirally disposed.

In accordance with yet other aspects of the invention, wherein handle surfaces are embossed on the circumferential surface. In a further embodiment, the fitment handle surfaces include a textured surface making grasping the fitment easy, secure and comfortable. An embodiment of the fitment includes an easy-to-grip collar configured to allow easy grasping of the device by either the user or a care-giver who may be providing assistance. Advantageously, the collar is selected to be large enough to be easily used by people with arthritis or other disabling or limiting infirmities. A further embodiment includes an arrow embossed on the circumferential surface.

As will be readily appreciated from the foregoing summary, the invention provides a urine collection receptacle configured for receiving a urine collection funnel in sealing engagement. The receptacle includes a bladder defining an orifice in the bladder. The orifice comprising a pair of opposed lips. A generally tubular fitment that has a circumferential surface, a first end and a second end, and an axis. The fitment defines a bore having a bore interior surface. The first end of the fitment is configured to receive a funnel in the bore in frictional engagement with the bore interior surface. The second end is configured to engage lips of a bladder at a lip interior surface in sealing engagement thereby configured to conduct a liquid through the funnel to the bladder.

### BRIEF DESCRIPTION OF THE DRAWINGS

The preferred and alternative embodiments of the present invention are described in detail below with reference to the following drawings.

FIG. 1a is a first isometric view of an inventive fitment;

FIG. 1b is a second isometric view of the inventive fitment;

FIG. 2 is an isometric view a urine collection funnel;

FIG. 3 is a plan view of a system including the urine collection funnel, the fitment, and a receiving bladder; and,

FIG. 4 is a plan view of the system including a collection stopper, the fitment, and the receiving bladder.

### DETAILED DESCRIPTION OF THE PREFERRED EMBODIMENT

A system for collecting urine includes a fitment for receiving a funnel in sealing engagement. The fitment includes a generally cylindrical tube having a circumferential surface, a first and a second end, and defining a bore having a bore interior surface. The first end is configured to receive a funnel in the bore; the funnel is in frictional engagement with the bore interior surface. The second end is configured to engage lips of a bladder at a lip interior surface in sealing engagement and thereby configured to conduct a liquid through the funnel to the bladder.

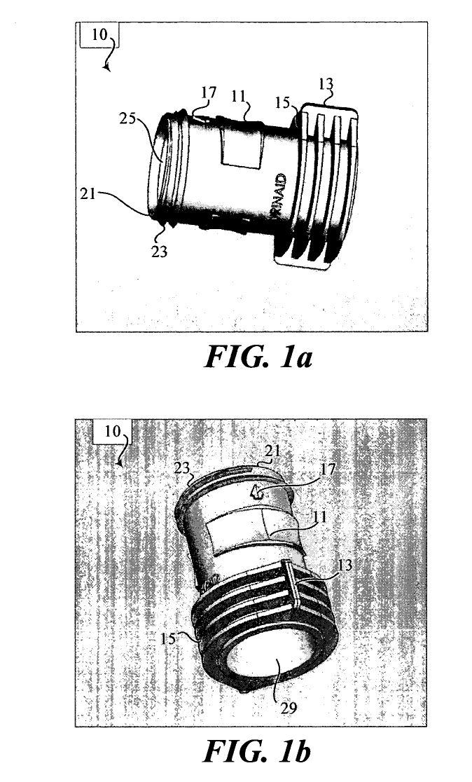

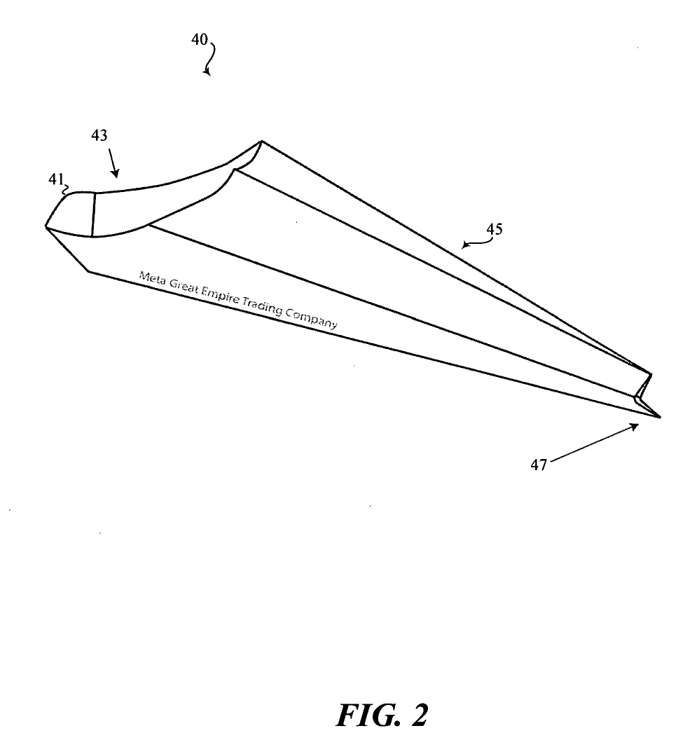

Referring to FIGS. 1a and 1b, a system for collecting urine from a human in a standing position includes a fitment 10. The fitment 10 is generally a cylindrical tube defining a bore 29, having a circular rim 21 at a first end. Within the bore 29, at the first end, at least one interior land 25 projects inward. In a preferred embodiment, the land is spirally configured to form a threaded surface.

At a second end, a pair of opposed fins 13 extend longitudinally, parallel to an axis of the tube, protruding radially from an outer surface generally at the second end.

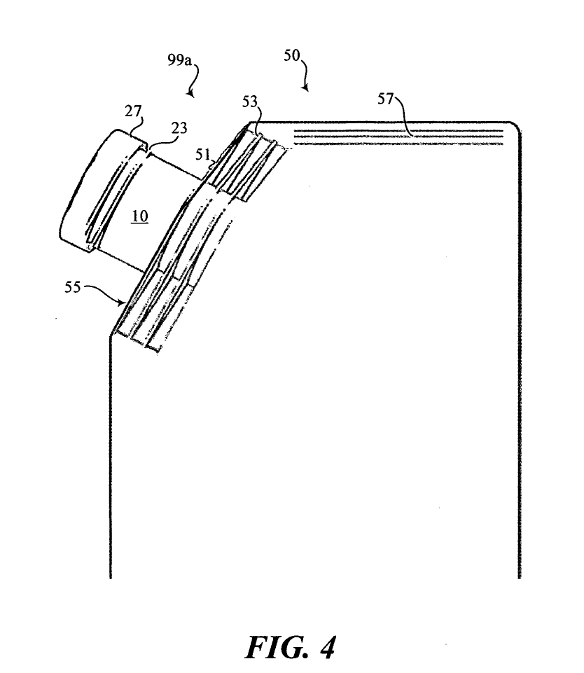

A plurality of lands 15 project orthogonally to the axis, extending between the opposed fins 13, the ribs 15 have generally ellipsoid profiles being configured to engage a plurality of formed ribs 53 (FIG. 3).

Approximately midway between the first and second ends a first and a second embossed handle surface 11 extends circumferentially around an exterior surface or barrel of the fitment 10. Optionally, an arrow is embossed to indicate an insertion port defined by the circular rim 21.

Additionally, an exterior land 23 extends around an outside of the barrel configured to engage a cover a corresponding interior land on a cover. In a preferred embodiment, the exterior land 23 spirals around the barrel to engage interior lands on a “twist off” cover (not shown). In a second embodiment, the exterior land 23 extends circularly to engage a circular interior on a “pop off” cover (not shown).

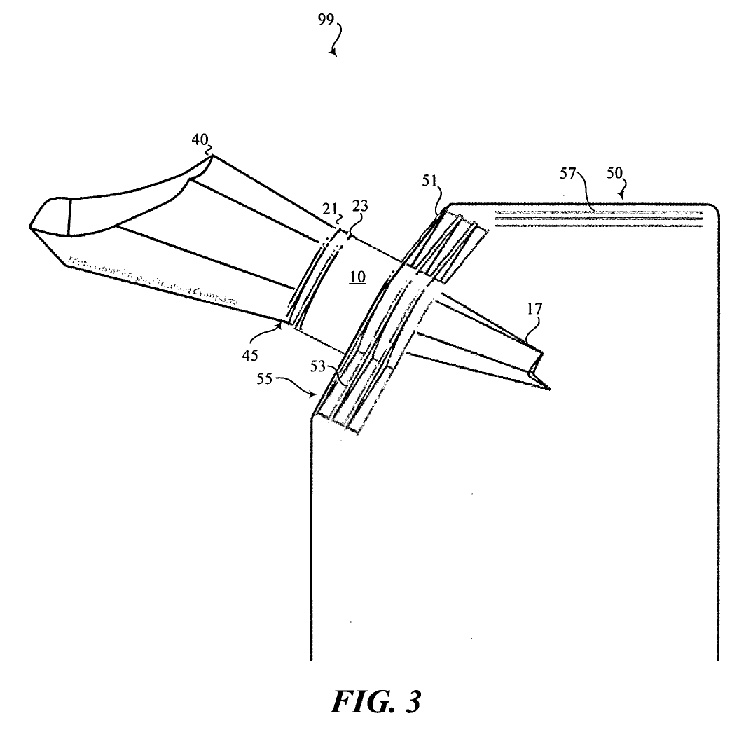

Referring to FIGS. 1a, 1b, and 2, a urine collection funnel 40 is configured to collect urine from a human in a standing posture. The urine collection funnel 40 includes a collection port 43 defined by a collection rim 41. Generally, the collapsible urine collection funnel is taught in U.S. Pat. No. 4,108,222 issued to Kaufman on Aug. 22, 1978 entitled “Collapsible Disposable Funnel” and incorporated in its entirety by this reference.

In a preferred embodiment of the invention, the urine collection funnel 40 comprises flexible panels 45 having fold lines defining boundaries of the panels 45, the panels 45 being foldable to define a substantially flat member in a first position and expansible to define a tapered passageway in a second position. The urine collection funnel 40 defines the collection port 43 having the collection rim 41. The collection port 43 is contoured to engage the female perineum area enclosing the urethra at the collection rim. A longitudinal axis of the urine collection funnel 40 is obliquely disposed to the collection port 43.

A conduit port 47 is situated in opposed relation to the collection port 43 and is configured to conduct urine collected through the collection port 43 through the urine collection funnel 40.

The flexible panels 45 are advantageously formed of a water-resistant material such as a coated fiberboard. In one embodiment of the invention, the water-resistant material is also biodegradable. Advantageously, material for forming the flexible panels 45 is selected to be suitably deformable.

Referring to FIG. 3, the urine collection funnel 40 is inserted into the bore 29 the fitment 10. The flexible panels 45, engage the interior land 25 (FIG. 1b). Deforming pressure exerted on the flexible panels 45 by the interior land 25 (FIG. 1b) locks the urine collection funnel 40 into secure coupling relation with the circular rim 21. The exterior land 23 is visible.

The second end of the fitment 10 engages a series of formed ribs 53 on an interior surface within lips 55 of a urine collection bladder 50 sealed in watertight engagement. The fins 13 (FIGS. 1a, 1b) urge against vertices 51 in the ribs 53. Engagement may either be by frictional engagement or, optionally, by fusing the ribs 53 to the interior surface of the lips 55.

An embodiment of the system includes a puncture and tear resistant bladder 50 of food-grade plastic suitable for safe handling and transport. The bladder 50 optionally includes a gusseted bottom panel (not shown) such that as the bladder expands filling with fluid, the gusseted bottom serves as a stable base such that the bladder is stable placed on the floor or other flat surface.

When the urine collection funnel 40 is engaged in the fitment 10, and the fitment 10 is, in turn, engaged in the urine collection bladder 50, the conduit port 17 extends to a point centrally located within the urine collection bladder 50 thereby avoiding clogging contact with walls of the urine collection bladder 50. In a preferred embodiment of the invention, hanging ribs 57 are formed into an edge of the urine collection bladder 50 to slidably engage in a slot (not shown) for suspension during transportation. The urine collection funnel 40 may be either slidably or twistingly disengaged based upon the configuration of the interior land 25 (FIG. 1b).

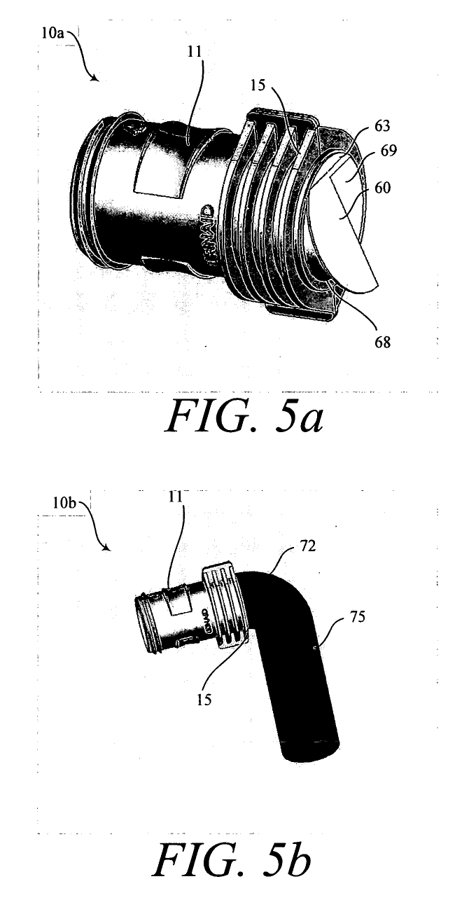

Referring to FIG. 4, the exterior land 23 is engaged in a second interior land (not shown) on an interior surface of sealing cover 27. The exterior land 23 may be either formed in spiral configuration or in circular configuration thus to engage in “twist off” or “pop off” sealing engagement respectively. The ribs 15 (FIGS. 1a, 1b) on the fitment 10 remain in watertight sealing engagement with the ridges 53 on an interior surface of lips 55 such that the vertices 51 of the lips 55 receive the fins 13, thereby holding the fitment in registration with the urine collection bladder 50. Hanging ribs 57 are suitably deployed for advantageous suspension from a slot.

Alternate embodiments are configured to prevent the system from allowing a reverse flow of urine through the fitment. Referring to FIG. 5a, a fitment 10a is configured to include a flapper valve 60 pivoting about a live hinge assembly 63. The flapper valve 60 is configured as a check valve such that the flapper valve 60 is urged into a closed position resting against a seat 68 raised in the outermost face of the outermost of the plurality of lands 15 the project orthogonally to the axis of the fitment 10a and a seat panel 69. As configured, the flapper valve 60 allows the flow of urine or other fluid through the fitment 10a in a generally downward direction to fill the urine collection bladder 50 (FIGS. 3, 4), but the flapper valve 60 closes if the fluid attempts to flow back through the fitment 10a by action of the live hinge assembly 63.

The flapper valve 60 is urged closed by the action of the live hinge assembly 63, the live hinge including linear molecules of a nylon or other suitable plastic used to cast the fitment 10a, aligned fairly accurately across the hinge, parallel to each other and perpendicular to an axis of the hinge. Live hinges are generally formed by flowing the material through a narrow gap at the site of the hinge, then the natural tendency to move the flapper valve 60 into a closed position is enhanced by stretching the plastic in a semi-molten phase (before the material is crystallised to a significant degree), by flexing the hinge as the plastic cools.

In operation, the fitment 10a is advantageous as it allows collection of urine in environments that might otherwise not allow the collection. For instance, where a patient is transported in an ambulance, vibration from movement over pavement may cause urine to tend to flow reversedly through the fitment 10a though securely supported at the embossed handle surface 11. Operation of the flapper valve 60 in concert with the live hinge assembly 63 assures that urine collected will not be spilled in the collection process.

Still another embodiment of the fitment 10b seeks to similarly provide the additional security of exploiting a check valve. Referring to FIG. 5b, a duckbill fitment 10b having an elbow 72 and a duckbill check valve 75 formed of an elastic rubber compound and fusedly attached at the outermost face of the outermost of the plurality of lands 15 the project orthogonally to the axis of the fitment 10b. Duckbill check valves 75 allow free flow with positive differential pressure. With negative differential pressure, backflow is checked by action of the duckbill check valve 75.

Duckbill check valves 75 can be designed to operate with low pressures of a magnitude of a few millimeters (inches) of H2O to several hundred kPa (psi), depending on valve size, geometry, and selected elastic rubber compound characteristics. Duckbill check valves 75 are also designed to start functioning at specific opening and closing pressure ranges. When such pressures are selected at or near the minimal pressures, the duckbill check valve 75 suitable allows urine to flow through the fitment 10b in a single direction to fill the urine collection bladder 50 (FIGS. 3, 4), while suitably preventing backflow.

In operation, the fitment 10b shares advantages with the fitment 10a (FIG. 5a) in that it too allows collection of urine in environments that might otherwise not allow the collection. Similarly, thus, where a patient is transported in an ambulance, vibration from movement over pavement may cause urine to tend to flow reversedly through the fitment 10a though securely supported at the embossed handle surface 11. Operation of the duckbill check valve 75 assures that urine collected will not be spilled in the collection process. The elbow 72 further assures that the direction of a flow of urine is not aligned with the axis of the fitment 10b. By suitably redirecting the urine flow, the elbow 72 acts as a baffle to reverse flow of urine, preventing splashing of the urine.

Additional check valve assemblies are also suitable, operation of which are known in the art. For instance, a captured ball assemble with a highly buoyant low density ball that works by allowing the denser urine to urge, by buoyancy or by urging of a suitably spring into engagement with a ball seat to prevent flow of the urine out of the urine collection bladder 50 (FIGS. 3, 4). This series of check valve fitments is given as non-limiting examples of the use of a check valve to prevent the flow of urine out of the urine collection bladder 50 (FIGS. 3, 4).

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A fitment for receiving a funnel in sealing engagement, the fitment comprising:
a generally cylindrical tube having a circumferential surface, a first and a second end, and an axis and defining a bore having a bore interior surface:
the first end being configured to receive a funnel in the bore in frictional engagement with the bore interior surface;
the second end being configured to engage lips of a bladder at a lip interior surface in sealing engagement thereby configured to conduct a liquid through the funnel to the bladder.

2. The fitment of claim 1, wherein the fitment includes at least one circumferential rib configured to engage at least one linear ridge formed on the lip interior surface.

3. The fitment of claim 2, wherein the at least one circumferential rib is frictionally engaged with the at least one linear ridge.

4. The fitment of claim 2, wherein the at least one circumferential rib is fusedly engaged with the at least one linear ridge.

5. The fitment of claim 1, wherein the fitment includes at least one axial fin configured to engage at least one vertex of the lips in order to maintain registration between the fitment and the lips.

6. The fitment of claim 1, wherein an internal land is disposed on the first internal surface generally at the first end, the internal land being configured to engage a funnel.

7. The fitment of claim 6, wherein the internal land is further configured to engage the funnel to sealingly engage flexible panels of the funnel against a fitment rim.

8. The fitment of claim 6, wherein the internal land is circularly disposed.

9. The fitment of claim 6, wherein the internal land is spirally disposed.

10. The fitment of claim 1, wherein an external land configured to sealingly engage a cover is disposed on the circumferential surface generally at the first end.

11. The fitment of claim 10, wherein the external land is circularly disposed.

12. The fitment of claim 10, wherein the external land is spirally disposed.

13. The fitment of claim 1, wherein handle surfaces are embossed on the circumferential surface.

14. The fitment of claim 1, wherein an arrow is embossed on the circumferential surface.

15. The fitment of claim 1, wherein a check valve is disposed at the first end, configured to allow the liquid through the fitment to the bladder and further configured to prevent a flow of the liquid out of the bladder through the fitment.

16. The fitment of claim 15, wherein the check valve includes a flapper valve.

17. The fitment of claim 15, wherein the check valve includes a duckbill check valve.

18. The fitment of claim 15, wherein the check valve includes a ball check valve.

19. The fitment of claim 1, wherein an elbow is disposed at the first end, configured to allow the liquid through the fitment to the bladder and further configured to prevent a splash of the liquid out of the bladder through the fitment.

20. A urine collection receptacle for receiving a urine collection funnel in sealing engagement, the receptacle comprising:
a bladder defining an orifice in the bladder, the orifice comprising a pair of opposed lips; and
a generally tubular fitment a having a circumferential surface, a first end and a second end, and an axis and defining a bore having a bore interior surface:
the first end being configured to receive a funnel in the bore in frictional engagement with the bore interior surface;
the second end being configured to engage lips of a bladder at a lip interior surface in sealing engagement thereby configured to conduct a liquid through the funnel to the bladder.

21. The receptacle of claim 20, wherein the fitment includes at least one circumferential rib configured to engage at least one linear ridge formed on the lip interior surface.

22. The receptacle of claim 21, wherein the at least one circumferential rib is frictionally engaged with the at least one linear ridge.

23. The receptacle of claim 21, wherein the at least one circumferential rib is fusedly engaged with the at least one linear ridge.

24. The receptacle of claim 20, wherein the fitment includes at least one axial fin configured to engage at least one vertex of the lips in order to maintain registration between the fitment and the lips.

25. The receptacle of claim 20, wherein an internal land is disposed on the first internal surface generally at the first end, the internal land being configured to engage a funnel.

26. The receptacle of claim 25, wherein the internal land is further configured to engage the funnel to sealingly engage flexible panels of the funnel against a fitment rim.

27. The receptacle of claim 25, wherein the internal land is circularly disposed.

28. The receptacle of claim 25, wherein the internal land is spirally disposed.

29. The receptacle of claim 20, wherein an external land configured to sealingly engage a cover is disposed on the circumferential surface generally at the first end.

30. The receptacle of claim 29, wherein the external land is circularly disposed.

31. The receptacle of claim 29, wherein the external land is spirally disposed.

32. The receptacle of claim 20, wherein handle surfaces are embossed on the circumferential surface.

33. The receptacle of claim 20, wherein an arrow is embossed on the circumferential surface.

34. The receptacle of claim 20, wherein the bladder comprises a gusseted panel.

35. The receptacle of claim 34, wherein the gusseted panel is configured to provide a base for the bladder.

36. The receptacle of claim 34, wherein the bag comprises a first and a second side panel and wherein the gusseted panel is configured to urge the first and the second side panels apart.

37. The receptacle of claim 20, wherein a check valve is disposed at the first end, configured to allow the liquid through the fitment to the bladder and further configured to prevent a flow of the liquid out of the bladder through the fitment.

38. The receptacle of claim 37, wherein the check valve includes a flapper valve.

39. The receptacle of claim 37, wherein the check valve includes a duckbill check valve.

40. The receptacle of claim 37, wherein the check valve includes a ball check valve.

41. The receptacle of claim 20, wherein an elbow is disposed at the first end, configured to allow the liquid through the fitment to the bladder and further configured to prevent a splash of the liquid out of the bladder through the fitment.
